(19) United States
(12) Patent Application Publication
(10) Pub. No.: US 2025/0344937 A1
(43) Pub. Date: Nov. 13, 2025

(54) SYSTEMS AND METHODS FOR DETECTING A HYDRATION CONDITION OF A FLEXBILE ELONGATED DEVICE

(71) Applicant: Intuitive Surgical Operations, Inc., Sunnyvale, CA (US)

(72) Inventors: Majeed A. Foad, Sunnyvale, CA (US); John Tang, San Jose, CA (US)

(21) Appl. No.: 19/201,545

(22) Filed: May 7, 2025

Related U.S. Application Data
(60) Provisional application No. 63/644,351, filed on May 8, 2024.

(51) Int. Cl. A61B 1/00; A61B 34/20; A61B 34/30; A61B 90/00; A61M 25/00

(52) U.S. Cl. CPC A61B 1/00057; A61B 1/00006; A61B 1/00055; A61B 34/20; A61B 2034/2061; A61B 2034/301; A61B 2090/064; A61B 2090/0807; A61B 2562/029; A61M 2025/0046

(57) ABSTRACT

A system may comprise a flexible elongated device including a flexible elongated body and a hydrophilic lubricious layer on the flexible elongated body. The system may also comprise a sensor system configured to detect a hydration indicator for the hydrophilic lubricious layer and a control system configured to evaluate the hydration indicator to determine a hydration condition of the hydrophilic lubricious layer.

### RELATED APPLICATIONS

This application claims priority to and benefit of U.S. Provisional Application No. 63/644,351 filed May 8, 2024 and entitled “Systems and Methods for Detecting a Hydration Condition of a Flexible Elongated Device,” which is incorporated by reference herein in its entirety. This patent application is related to U.S. Provisional Patent Application 63/644,608, entitled “SYSTEMS AND METHODS FOR HYDRATING A FLEXIBLE ELONGATED DEVICE,” filed May 8, 2024 and U.S. Provisional Patent Application 63/644,404, entitled “FLEXIBLE ELONGATED DEVICE WITH A LUBRICIOUS LAYER AND METHODS OF USE”, filed May 8, 2024 which are incorporated by reference herein in their entirety.

### FIELD

The present disclosure relates to detecting hydration indicators, and more particularly to systems and methods for detecting a hydration condition of a lubricious coating disposed on the flexible elongated device.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert a minimally invasive medical instrument (including surgical, diagnostic, therapeutic, and/or biopsy instruments) to reach a target tissue location. One such minimally invasive technique is to use a flexible elongated device, such as a flexible catheter, bronchoscope, or endoscope, which can be inserted into anatomic passageways and navigated toward a region of interest within the patient anatomy. When navigating passageways within a patient anatomy, the flexible elongated device may experience friction with the passageway walls resulting in difficulty navigating the device and reaching a target anatomical location. Systems and methods to measure and/or monitor indicia of lubricity of flexible elongated devices are needed.

### SUMMARY

The following presents a simplified summary of various examples described herein and is not intended to identify key or critical elements or to delineate the scope of the claims.

In some examples, a system may comprise a flexible elongated device including a flexible elongated body and a hydrophilic lubricious layer on the flexible elongated body. The system may also comprise a sensor system configured to detect a hydration indicator for the hydrophilic lubricious layer and a control system configured to evaluate the hydration indicator to determine a hydration condition of the hydrophilic lubricious layer.

In some examples, a method may comprise detecting, with a sensor system, a hydration indicator for a hydrophilic lubricious layer on a flexible elongated body of a flexible elongated device and evaluating, with a control system, the hydration indicator to determine a hydration condition for the hydrophilic lubricious layer.

In some examples, a non-transitory machine-readable media stores instructions that, when run by one or more processors, cause the one or more processors to: detect, with a sensor system, a hydration indicator for a hydrophilic lubricious layer on a flexible elongated body of a flexible elongated device and evaluate the hydration indicator to determine a hydration condition for the hydrophilic lubricious layer.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 illustrates an instrument system including a flexible elongated device with a lubricious layer, according to some examples.

FIGS. 2A-2C show cross sectional views of a lubricious layer at various hydration conditions, according to some examples.

FIG. 3 illustrates a manipulator assembly with a flexible elongated device extending into a patient anatomy, according to some examples.

FIG. 4 is a flowchart illustrating a method for detecting a hydration condition of a flexible elongated device, according to some examples.

FIG. 5 illustrates an instrument system including a flexible elongated device with a lubricious layer, according to some examples.

FIG. 6 illustrates an instrument system including a flexible elongated device with a lubricious layer, according to some examples.

FIG. 7 illustrates an instrument system including a flexible elongated device with a lubricious layer, according to some examples.

FIG. 8 illustrates an example of a flexible elongated device in a patient anatomy near a target tissue, according to some examples.

FIG. 9 is a simplified diagram of a medical system, according to some examples.

FIG. 10A is a simplified diagram of a medical instrument system, according to some examples.

FIG. 10B is a simplified diagram of a medical instrument including a medical tool within a flexible elongated device, according to some examples.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

The systems described herein may include flexible elongated devices (e.g., catheters, bronchoscopes, or endoscopes) that include a lubricious layer. As flexible elongated devices navigate anatomic passages, they may experience friction with the inner wall of the passageways or with components of manipulator systems to which they may be attached. Friction may compromise control and navigation of the flexible elongated device through increased resistance, stick-slip behavior, prolapse of the device externally or internally of the patient, or an inability to reach a target location. Flexible elongated devices may be coated with a lubricious layer or coating, such as hydrophilic coating, to reduce or eliminate the issues associated with friction. In some types of anatomic passageways, such as cardiovascular or neurovascular passageways, naturally-present body fluids or mucous may activate a lubricious layer of a flexible elongated device to augment lubrication provided by the anatomy. In contrast, flexible elongated devices navigating through other types of anatomic passageways (e.g., lung passageways) may be exposed to flowing air that dries the flexible elongated devices and increases the coefficient of friction of the flexible elongated device. Lubricious layers activated by non-anatomic sources of hydration may be used to increase lubrication in these drier anatomic environments. The systems described herein may be used to measure and/or monitor lubricity of flexible elongated devices based on indicia of hydration levels of a lubricious layer disposed on the flexible elongated device.

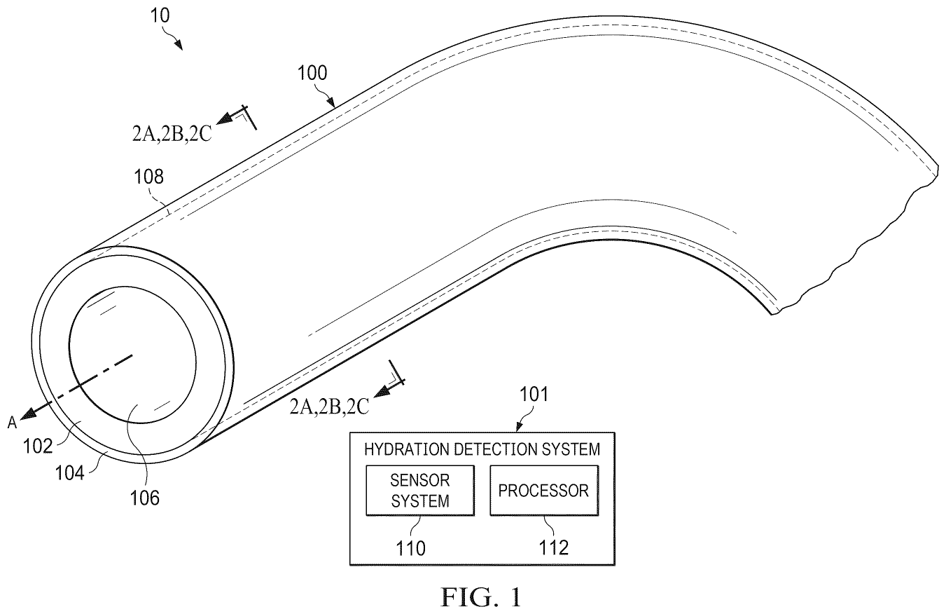

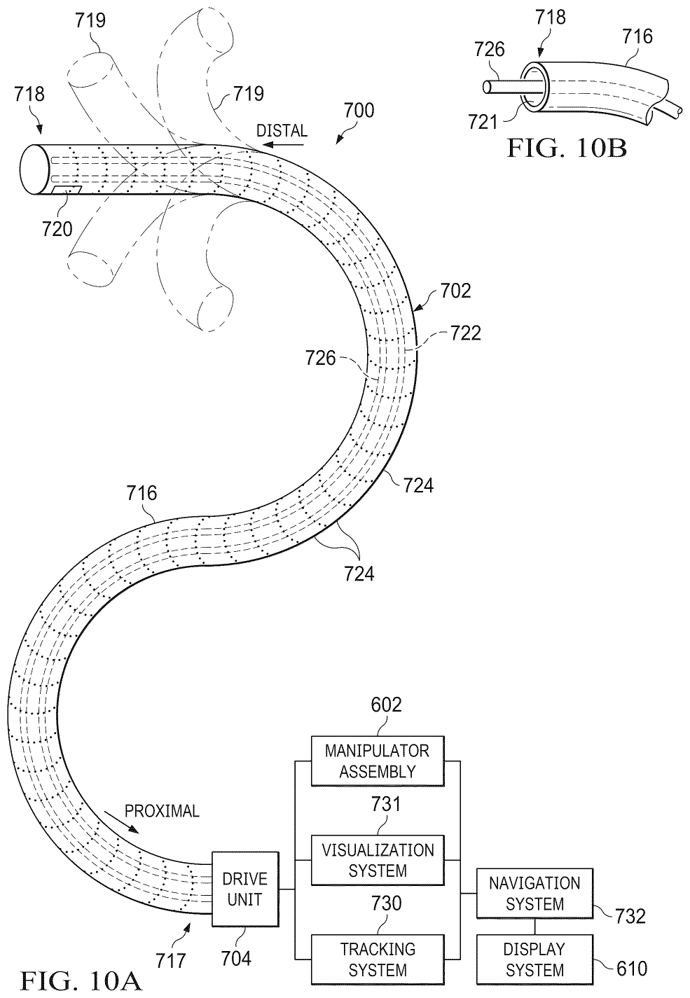

FIG. 1 illustrates a system 10 including a flexible elongated device 100 and a hydration detection system 101. The flexible elongated device 100 includes a flexible elongated body 102 and a lubricious layer 104 extending along at least a portion of the length of an outer surface 108 of the body 102. The flexible elongated body 102 may extend along a longitudinal axis A and define a lumen 106 through which, for example, tools may be inserted or fluids may be introduced or evacuated. In some examples, the flexible elongated device 100 may be a component of a robotically assisted medical instrument system or a manually-controlled medical instrument system that controls articulation and insertion/retraction of the flexible elongated device 100. An example of a medical instrument system including a flexible elongated device that is bendable and steerable in multiple degrees of freedom is described below in FIGS. 10A and 10B (e.g., system 700).

The lubricious layer 104 may include, for example, a hydrophilic substance that may promote lubricity thereby reducing or preventing stick-slip or irregular sliding behaviors as the flexible elongated device 100 is introduced into a patient anatomy. The lubricious layer 104 may be applied to an outer surface 108 of the flexible elongated body 102 for example by dip coating, spray-on application, wrap material application, wipe-on application, or as a tubular overlay. In some examples, the lubricious layer 104 may extend along a portion of the outer surface less than the entire length of the flexible elongated body. For example, the lubricious layer may extend along approximately six inches of the distal end portion of the flexible elongated body 102. In some examples, a hydrophilic lubricious layer may have a hydrated or activated condition in which the hydrophilic material is hydrated and retentive of fluid, and the hydrophilic lubricious layer may have an anhydrous or inactivated condition in which the hydrophilic material is anhydrous or dehydrated. In some examples, the lubricious layer 104 may include a visible pigment or dye that imparts an identifying color to the lubricious layer. The visible pigment may identify which portion of the flexible elongated device include the lubricious layer and thus will transition to a hydrated condition when hydrated. The visible pigment may also or alternatively indicate wear or delamination of the lubricious layer, signaling for example that the lubricious layer should be reapplied or the device should be discarded. In some alternative examples, the lubricious layer may include a hydrophobic material.

The hydration detection system 101 includes a sensor system 110 configured to detect a hydration indicator for the lubricious layer 104. The hydration detection system may also include at least one processor 112 for evaluating the hydration indicator to determine the hydration condition (e.g., level of hydration) of the flexible elongated device 100 or the lubricious layer 104 disposed thereon. A hydration condition may include, for example, a fully hydrated condition, a threshold hydrated condition, a sub-threshold hydrated condition, an anhydrous condition, as described below. The sensor system 110 may include any of a variety of sensors including time-based sensors, force sensors, shape sensors, optical sensors, humidity sensors, imaging systems, light sensors, and/or vibration sensors for detecting indicia of a hydration condition of the lubricious layer 104. The sensor system 110 may include components located in or on the flexible elongated device 100 and/or components located on other structures in a patient environment such as a manipulator assembly (e.g., manipulator assembly 200), an anatomic orifice device (e.g., anatomic orifice device 206), or the like. The processor 112 may be a processor of a robot-assisted medical system (e.g. a processor of control system 612). Although the processor 112 is shown as a single block in FIG. 1, the processor 112 may include two or more separate data processing circuits with portions of the processing being performed in different locations. Although certain examples of the hydration detection system 101 are described separately, sensor systems may be combined or used together.

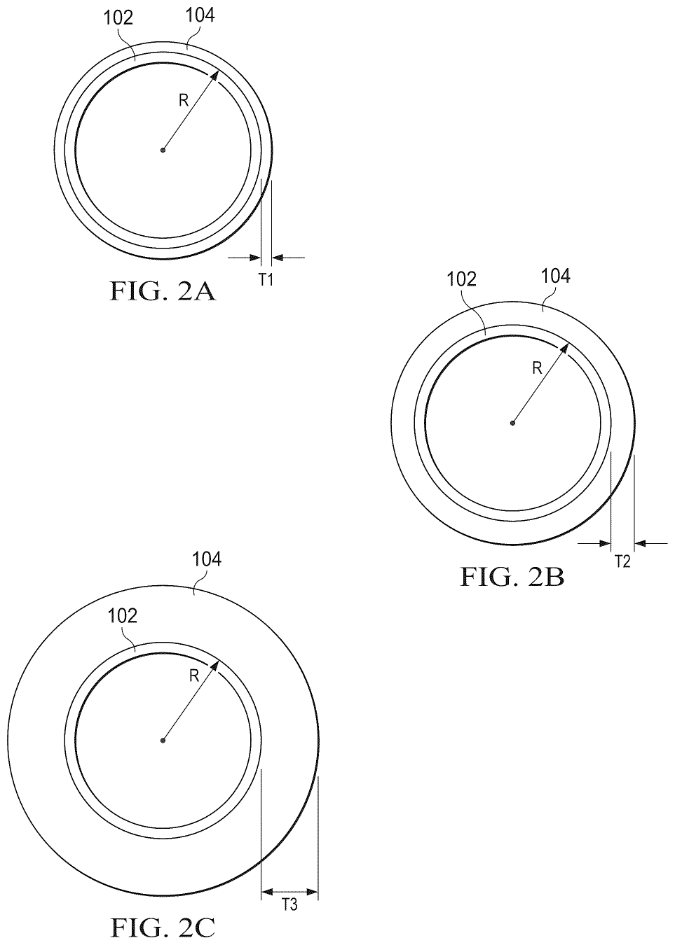

FIG. 2A illustrates a cross-sectional view of the flexible elongated device 100 with a hydrophilic lubricious layer 104 in an anhydrous or dehydrated condition. FIG. 2B illustrates a cross-sectional view of the flexible elongated device 100 with the hydrophilic lubricious layer 104 in a threshold hydrated condition. FIG. 2C illustrates a cross-sectional view of the flexible elongated device 100 with the hydrophilic lubricious layer 104 in a fully hydrated condition. FIGS. 2A, 2B, and 2C are not intended to be drawn to scale. The flexible elongated body 102 may have a radius R. As initially deposited on the flexible elongated body 102 and/or in the anhydrous condition, the lubricious layer 104 may have a thickness T1 as shown in FIG. 2A. As the lubricious layer 104 is exposed to a hydrating fluid such as water, saline, human anatomic fluid/mucous, or another hydrating liquid or gas, the lubricious layer 104 may swell to a thickness T2 in a threshold hydrated condition, as shown in FIG. 2B. The threshold hydrated condition may be, for example, a minimum hydration condition for a particular system or anatomic environment. In a fully hydrated condition, the lubricious layer 104 may swell to a thickness T3. In some examples, the thickness T1 in the anhydrous condition may measure between approximately 2 and 3 μm, and the thickness T3 in the fully hydrated condition may measure between approximately 20 and 25 μm. The thickness T2 may be a thickness between the thicknesses T1 and T3.

Hydrophilic properties of the lubricious layer 104 may allow the hydrophilic coating to absorb the hydrating fluid. When the lubricious layer 104 is in the fully hydrated condition, a coefficient of friction of the flexible elongated device 100 may be low. As the lubricious layer dehydrates and transitions to the anhydrous condition, the lubricity of the flexible elongated device 100 may decrease and the coefficient of friction may increase. The hydration condition and the corresponding coefficient of friction may affect the level of precision and control with which the flexible elongated device 100 can be maneuvered in the patient anatomy. As the coefficient of friction increases, the flexible elongated device 100 may experience greater resistance, which can lead to undesirable outcomes, for example, stick-slip behavior.

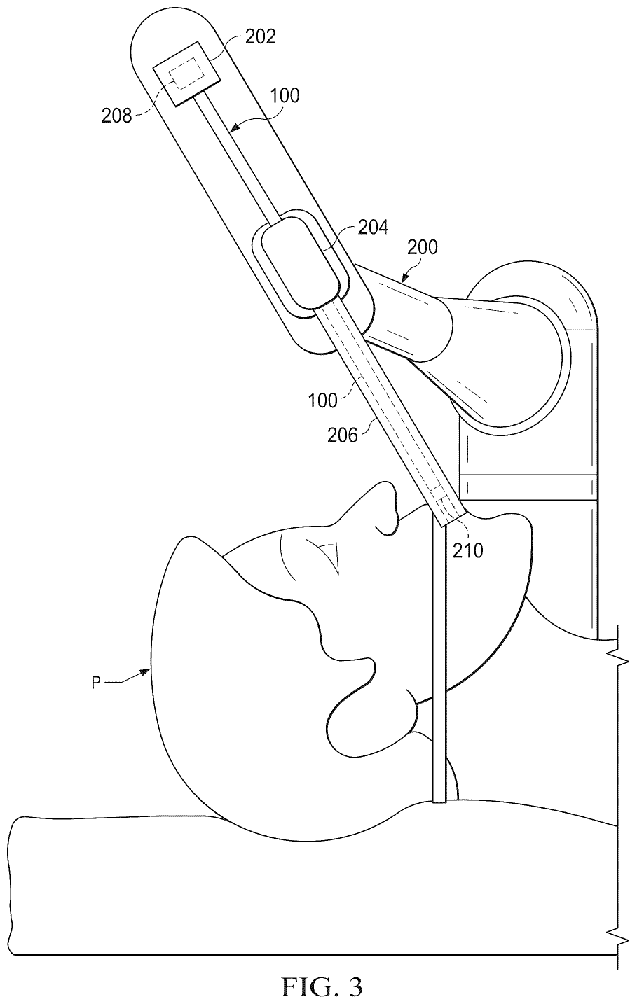

FIG. 3 illustrates a manipulator assembly 200 connected to the flexible elongated device 100. The manipulator assembly 200 may be a robotically-assisted manipulator assembly. For example, the manipulator assembly 200 may be a component (e.g., the manipulator assembly 602) of a robotically-assisted manipulator system (e.g., the robotically-assisted manipulator system 600). The manipulator assembly 200 may include an instrument carriage 202 to which a proximal end of the flexible elongated device 100 is connected. The manipulator assembly 200 may include a connector device 204 to which the flexible elongated device 100 may be coupled. In some examples, the connector device 204 may swivel or rotate relative to the manipulator assembly 200. The flexible elongated device 100 may extend through an anatomic orifice device 206 and into the patient anatomy P. The anatomic orifice device 206 may be, for example, an endotracheal tube, a laryngeal mask airway, or a cannula, and may be fixed to patient anatomy P to facilitate insertion of various medical devices. In some examples, the instrument carriage 202 or other components of the manipulator assembly 200, such as the connector device 204, may include a sensor 208 for measuring a property or characteristic of the flexible elongated device 100. For example, the sensor 208 may be a force sensor that measures forces applied to the flexible elongated device 100. In some examples, the force sensor 208 may be located in or on the flexible elongated device 100. In some examples, the sensor 208 is optional. In some examples, the anatomic orifice device 206 may include a sensor 210 for measuring a property or characteristic of the flexible elongated device 100. For example, the sensor 210 may be an optical sensor or photodiode for measuring a color change in the flexible elongated device. In other examples, the sensor 210 may include a light sensor. In other examples, the sensor 210 may include a vibration sensor. In some examples, the sensor 210 is optional.

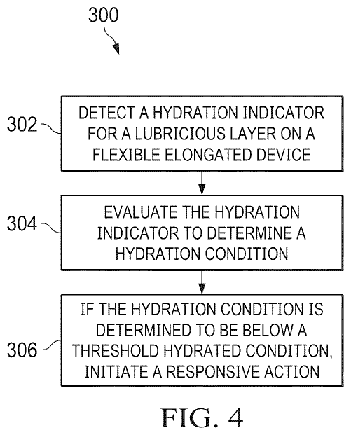

FIG. 4 is a flowchart illustrating a method 300 for detecting a hydration condition or level of a lubricious layer of a flexible elongated device. In some examples, if the hydration condition meets a minimum threshold hydrated condition, an operator and/or a robot-assisted system may initiate or continue with a procedure using the flexible elongated device. If the hydration condition is anhydrous or below a minimum threshold hydrated condition, an operator and/or a robot-assisted system may initiate an action to, for example, prevent injury to a patient, prevent damage to the flexible elongated device, or improve control of the flexible elongated device. The method 300 is illustrated as a set of operations or processes that may be performed in the same or in a different order than the order shown. One or more of the illustrated processes may be omitted in some examples of the method. Additionally, one or more processes that are not expressly illustrated in FIG. 4 may be included before, after, in between, or as part of the illustrated processes. In some examples, one or more of the processes of method 300 may be implemented, at least in part, by a control system executing code stored on non-transitory, tangible, machine-readable media that when run by one or more processors (e.g., the processors of a control system 612) may cause the one or more processors to perform one or more of the processes.

At a process 302, a hydration indicator may be detected for a hydrophilic lubricious layer on a flexible elongated device. For example, a hydration indicator may be detected for the lubricious layer 104 of the flexible elongated device 100 using the hydration detection system 101 including the sensor system 110. As described below, the sensor system 110 may include any of a variety of sensors alone or in combination. Such sensors may include time-based sensors, force sensors, shape sensors, optical sensors, humidity sensors, imaging systems, light sensors, and/or vibration sensors for detecting indicia of hydration of the lubricious layer 104. In other examples, an operator or other human can detect the hydration indicator. In some examples, the detection of the hydration condition may occur while at least a portion of the lubricious layer 104 is extended within the patient anatomy. In some examples, the detection of the hydration condition may occur while the flexible elongated device 100 and/or the lubricious layer 104 is external to the patient anatomy.

At a process 304, the hydration indicator may be evaluated to determine a hydration condition. For example, a hydration indicator detected for the lubricious layer 104 using the hydration detection system 101 may be evaluated to determine if the lubricious layer is in an anhydrous condition (FIG. 2A), a fully hydrated condition (FIG. 2C), or meets a threshold hydrated condition (FIG. 2B). The hydration indicator may include, for example, a duration, a force, a shape sensor reading, an optical characteristic, a humidity level, an object in an image, a light quality, and/or a vibration level. The evaluation may be performed, for example by the processor 112 of the hydration detection system 101 on another processor such as a processor of a control system (e.g. control system 612). In other examples, an operator or other human can perform the evaluation.

At a process 306, if the hydration condition for the lubricous layer is determined to be below a threshold hydrated condition, a responsive action may be initiated. The process 306 may be optional. In some examples, if the evaluation of hydration indicator for the lubricious layer 104 results in a determination that a current hydration condition is below a threshold hydrated condition, an action such as issuing an alert, changing a motion of the flexible elongated device 100, and/or delivering hydration to the flexible elongated device 100 may be initiated. In some examples, a combination of actions may be initiated based on the determination that the lubricious layer is below a threshold hydrated condition.

In some examples, an alert may be a visual warning displayed on a display system (e.g. the display system 610), an auditory alarm or message, tactile feedback via an operator input device (e.g. the operator input device of master assembly 606), or form of feedback perceptible to an operator of the flexible elongated device 100. The alert may provide a warning about potential injury that may be caused by further operation of the flexible elongated device 100 in a sub-threshold hydrated condition, instructions for hydrating the lubricious layer 104, or other information relevant to the safety or efficacy of flexible elongated device 100. Based on the alert an operator may take an appropriate response such as pausing the procedure, retracting the flexible elongated device 100, rehydrating the lubricious layer 104, or altering the speed with which the flexible elongated device 100 is being inserted or retracted.

In some examples, the initiated action may include changing a motion of the flexible elongated device. For example, a robot-assisted medical system (e.g. system 600) may suspend, slow, or otherwise alter a commanded motion if the lubricious layer 104 is insufficiently hydrated. In other examples, the robot-assisted medical system may retract or rotate the flexible elongated device 100. In some examples, the robot-assisted medical system may prevent an initial insertion of the flexible elongated device 100 in a sub-threshold hydrated condition.

In some examples, the initiated action may include delivering hydration to the lubricious layer 104. Examples of systems and methods for delivering hydration are described, for example, in U.S. Provisional Patent Application 63/644,308, entitled “SYSTEMS AND METHODS FOR HYDRATING A FLEXIBLE ELONGATED DEVICE,” filed May 8, 2024, which is incorporated by reference herein in its entirety. For example, a hydrating fluid may be delivered through the lumen 106 of the flexible elongated device 100. In other examples, a hydrating fluid may be delivered into the patient anatomy alongside the flexible elongated device 100. In other examples, the flexible elongated device 100 may be retracted from the patient anatomy, hydrated, and returned to the patient anatomy. In some examples, the amount of hydrating fluid delivered to the flexible elongated device may be based on the level of the hydration condition, with more hydration delivered to a fully anhydrous lubricious layer and less hydration delivered to a merely moderately dehydrated lubricious layer.

The sensor system 110 used in the detection of the hydration condition may include any of a variety of sensors. In some examples, the sensor system 110 may include a timer, clock, or other device for measuring a duration of time. The timer may be configured to measure or determine an elapsed time that the flexible elongated device 100 and/or the lubricious layer 104 has been in an exposed environment. An exposed environment may be a dry or minimally hydrated environment including the ambient environment of a medical clinic, an environment external to a package in which a hydrated flexible elongated device is sealed, or a low or minimally hydrated anatomic environment such as a lung passageway. In an exposed environment, the lubricious layer 104 may dehydrate over time due to insufficient environmental hydration to maintain a threshold hydrated condition. An unexposed environment may be an environment that maintains a hydration condition of the lubricious layer generally above the threshold hydrated condition. An unexposed environment may include a sealed environment that prevents dehydration of the lubricious layer 104 below the threshold hydrated condition or a hydrated environment with sufficient environmental hydration to maintain the lubricious layer 104 generally above the threshold hydrated condition. As an example, if the flexible elongated device 100 is coated with the hydrophilic lubricious layer 104 and hydrated before the flexible elongated device 100 is packaged, the flexible elongated device 100 will remain in an unexposed environment until the package is opened for use. In some examples, a hydration fluid may be applied to a hydrophilic lubricious layer 104 while the flexible elongated device 100 is in an exposed environment such as a clinical environment.

Once a lubricious layer 104 in a hydrated condition enters an exposed environment (e.g., a sealed package is opened to an exposed environment or a hydrating fluid is applied to a lubricious layer in an exposed environment), the timer may begin measuring the amount of time that elapses thereafter. The timer may be automatically triggered by a sensed occurrence such as the opening of a package or the dispensing of a hydration fluid or may be manually triggered by personnel in the clinical environment. The timer may pause the measuring of the elapsed time if the flexible elongated device 100 transitions from an exposed environment to an unexposed environment. The elapsed time or duration of time in the exposed environment may be a hydration indicator for the lubricious layer 104. For example, the rate at which the lubricious layer dehydrates from a fully hydrated condition to the threshold hydrated condition may be modeled such that a duration of time in the exposed environment corresponds with a known or expected hydration condition or level. Thus, using the elapsed time as a hydration indicator, the hydration detection system 101 may the evaluate the known or expected hydration condition of the lubricious layer 104 by referencing the model.

A time-based dehydration model may have a linear or non-linear relationship between elapsed time and hydration condition. The model may also be adjusted based on hydration factors such as the humidity level in the clinical environment, the type of anatomic environment (e.g. fluid-filled or non-fluid-filled), the initial hydration level of the lubricious layer 104, patient-specific characteristics such as age and disease state, the type of initial hydration fluid applied to the lubricious layer 104, or other factors that may impact the hydration condition of the lubricious layer at a measured elapsed time in the exposed environment. The dehydration model may be used to detect past and future hydration conditions of the lubricious layer 104.

In some examples, a dehydration model may be created under various environmental conditions by tracking a change in thickness of a hydrophilic lubricious layer over a range of elapsed times. As shown in FIG. 2C, the lubricious layer 104 may have a thickness T3 in a fully hydrated condition. The thickness T3 may be measured at a time before the lubricious layer 104 has been exposed to air (e.g., in an unexposed condition) or immediately after transitioning to an exposed condition. The thickness of the lubricious layer 104 may decrease over time as the dehydration process occurs. For example, a measured elapsed time may correspond to the transition from the fully hydrated condition at a lubricious layer thickness for T3 and the threshold hydrated condition at the lubricious layer thickness T2. Pre-operative tests performed to create the dehydration model may use any number of measurements at any number of times throughout hydration cycle. The lubricious layer thickness may be measured using a variety of devices. In one example, a laser micrometer may be used to measures the thickness. In other examples, images of the flexible elongated device 100 with the lubricious layer 104 disposed thereon may be captured and the images can be analyzed to determine the changing thickness of the lubricious layer. In some examples, the pre-operative tests used to build the dehydration model for the lubricious layer 104 may be performed in an environment which simulates the environmental factors expect during an actual procedure. For example, the humidity or other environmental factors surrounding the lubricious layer 104 during the pre-operative bench test may be similar to the expected humidity or factors present during a procedure. Further, it is understood that the dehydration model for the lubricious layer 104 may change throughout various stages of a procedure. For example, one dehydration model could be calculated for use when the flexible elongated device 100 is exposed to the air within an operating room, but not yet inserted within the anatomic orifice device 206. Another dehydration model could be created for use when the flexible elongated device 100 is disposed within the anatomic orifice device 206 but not yet inserted into the patient anatomy. Yet another dehydration model could be formulated for use when the flexible elongated device 100 has been inserted into the patient anatomy. It is understood that different anatomic passageways within the patient anatomy would result in different rates of dehydration. The dehydration models can be created with these differences in mind. Even further, a single dehydration model may combine multiple dehydration models so that the single consolidated dehydration model can be used. Although pre-operative tests are described above, similar tests can be performed intra-operatively to determine a dehydration model using similar techniques as described above.

In some examples, the sensor system 110 may include the sensor 208 which may be a force sensor. The force sensor 232 may be a load cell configured to obtain friction force data based on the resistance observed by the manipulator assembly 200 and/or the instrument carriage 202 while the flexible elongated device 100 is inserted or retracted. As previously described, when the lubricious layer 104 is hydrated at or greater than the threshold hydrated condition, the coefficient of friction of the flexible elongated device 100 may be lower than the coefficient of friction when the lubricious layer in in an anhydrous condition. A higher coefficient of friction creates more friction and resistance between the flexible elongated device 100 and the patient anatomy or the components (e.g., connector device 204 or anatomic orifice device 206) which engage the flexible elongated device 100.

Friction force data from the load cells of the force sensor may be a detected hydration indicator evaluated by the hydration detection system 101 to determine the hydration condition (e.g., level of hydration) of the lubricious layer 104. Evaluating the hydration indicator may include recognizing a friction force pattern in the friction force data and comparing the recognized friction force patter to known patterns associated with various hydration conditions. For example, larger measured friction forces may correspond to anhydrous or sub-threshold hydrated condition. Smaller measured forces may correspond to less friction and above threshold or fully hydrated conditions. Force models may be determined in a pre-operative setting to establish relationships between measured forces and known or expected hydration conditions. The hydration detection system 101 may consider other factors present at the time the hydration condition is determined. For example, the hydration detection system 101 may know the insertion distance or other location criteria for the flexible elongated device 100. Location criteria may be useful to help determine whether friction and resistance are caused by an anhydrous condition of the lubricious layer or by the tortuous or narrow passageway is more distant anatomic areas of the patient anatomy such as more distal lung passageways. The measured friction data may be supplemented with other types of data to help accurately determine the hydration condition. The supplemental data may include, but is not limited to, procedure and patient-specific data, humidity data, and data relating to the anatomic passageway being navigated.

In some instances, the hydration detection system 101 may be configured to recognize insertion force patterns in the friction force data. Certain friction force patterns may be associated with stick-slip behavior by the flexible elongated device 100 or another occurrence affecting the procedure such as a physical obstruction in the anatomic passageway preventing insertion of the flexible elongated device 100. Similarly, the friction force patterns may be associated with typical friction forces felt when navigating certain anatomical passageways. The friction force patterns can be obtained pre-operatively in simulated environments, can be friction force patterns from prior experiments, or can be determined by simulating the procedure using a friction force model/equation.

In some scenarios, use of friction force data alone may result in a false hydration indicators. For example, the hydration detection system 101 may mistake a friction force pattern that results from a physical obstruction such as a tortuous turn for an indication that the flexible elongated device 100 is in an anhydrous condition. To prevent such false positives, the hydration detection system 101 may supplement friction force hydration indicia with other hydration indicia from other types of sensor systems, such as an image from an imaging element that identifies a physical obstruction. Alternatively shape sensor data, as described below, may supplement friction force data.

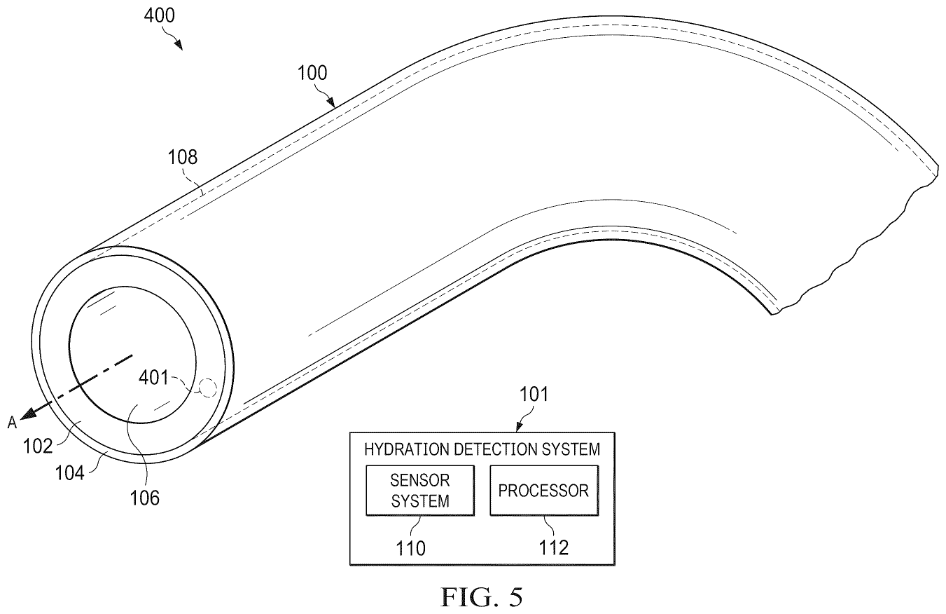

In some examples and with reference to FIG. 5, a system 400 may include the sensor system 110 which may include a shape sensor 401. The shape sensor 401 (e.g. the shape sensor 722) may include an optical fiber aligned with the flexible elongated body 102. The optical fiber of the shape sensor 401 may form a fiber optic bend sensor for determining the shape of flexible body 102. The shape sensor 401 may be used to identify buckling and unexpected shapes of the flexible elongated device 100. In this example, the detected shape of the flexible elongated device 100 may be the hydration indicator. The hydration detection system 101 can evaluate the shape (e.g., buckling, prolapse, or an unexpected shape) of the flexible elongated device 100 to determine the hydration condition of the lubricious layer 104. For example, the presence of a prolapse shape, buckling, or other characteristic shapes may correspond with a determination that the hydrophilic lubricious layer 104 is in a sub-threshold hydrated condition and the associated resistance within the patient anatomy has resulted in the detected buckled shape.

In some examples, the hydration detection system 101 can supplement shape sensor hydration indicia with other hydration indicia from other types of sensor systems such as an image from an imaging clement that identifies a physical obstruction. In some examples, the hydration detection system 101 may distinguish a shape profile (e.g. prolapse or buckle) associated with the presence of an obstruction from a shape profile (e.g., less pronounced shaped distortion) associated with friction from lack of hydration.

In some examples, a hydrophilic lubricious layer and/or the flexible elongated body may include a hydration indicator in the form of a color property of a hydrochromic pigment, and the sensor system 110 may include a sensor for detecting the hydrochromic pigment. For example, the sensor 210 may include an imaging system (e.g., imaging system 421) or an optical sensor configured to detect a hydrochromic pigment on the flexible elongated device 100. In various examples, the hydrochromic pigment may be incorporated into the lubricious layer 104, disposed under a transparent or semi-transparent lubricious layer 104, or deposited on an outer surface of the lubricious layer 104. The hydrochromic pigment may change color, shade, intensity, translucency, or another color property in the presence or absence of moisture. For example, the hydrochromic pigment 408 may have a first color when the lubricious layer 104 is in a fully hydrated condition, a second color when the lubricious layer 104 is in the threshold hydrated condition, and a third color when the lubricious layer 104 in the anhydrous condition.

The hydrochromic pigment may be detected in a variety of ways. For example, if the sensor 210 is an imaging system such as a camera, the sensor 210 may capture an image of a portion of the flexible elongated device 100 including the hydrochromic pigment, and the hydration detection system 101 may process the image data to evaluate the qualities (e.g., color, shade, intensity, translucency) of the hydrochromic pigment to determine the associated hydration condition of the lubricious layer 104. As another example, if the sensor 210 is an optical sensor such as a photodiode, the optical sensor may detect a color, translucency, or other characteristic of the hydrochromic pigment and the hydration detection system 101 may process the color data to evaluate the characteristics (e.g., color, shade, intensity, translucency) of the hydrochromic pigment and determine an associated hydration condition of the lubricious layer 104.

In some examples, the hydrochromic pigment sensor may be located on the anatomic orifice device, a manipulator assembly, in another location in the clinical environment, or on another instrument extended into the patient anatomy. In some examples, the hydrochromic pigment may be visually observed by an operator without use of a sensor system. For example, when the hydrochromic pigment is disposed on a proximal portion of the flexible elongated device 100, the hydrochromic pigment can be observed by an operator before the portion of the lubricious layer including the hydrochromic pigment enters the anatomic orifice device 206. Alternatively, the operator may observe the color the of the hydrochromic pigment through a window or opening in the anatomic orifice device 206. In some examples the operator may not observe the hydrochromic pigment directly, but rather, indirectly using photo and video capabilities. For example, a camera may be positioned in the clinical environment and the operator can view the color or a characteristic of the hydrochromic pigment on a display screen. The hydrochromic pigment may serve as a visual check as a procedure begins to ensure that the lubricious layer is sufficiently hydrated to initiate a procedure with the flexible elongated device 100.

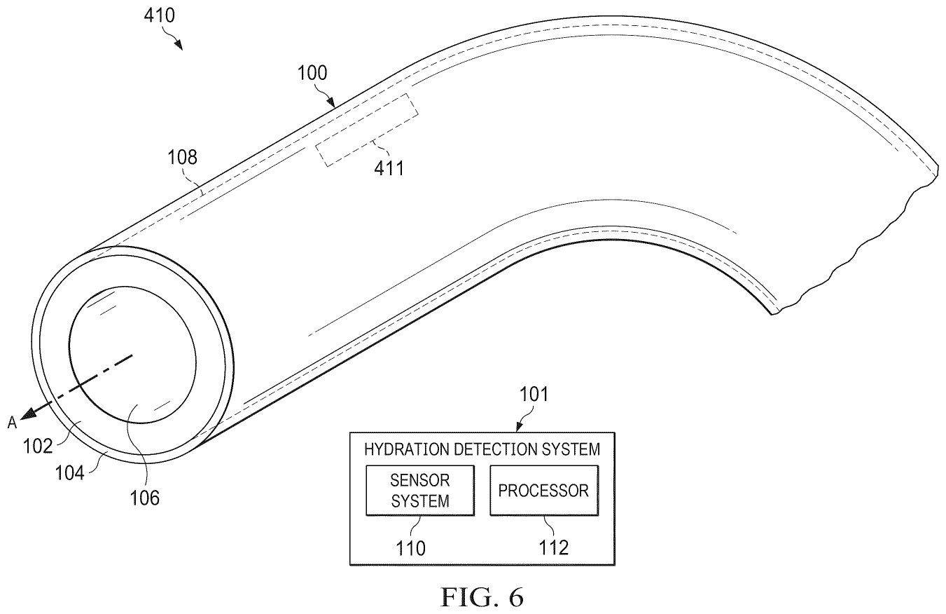

In some examples and with reference to FIG. 6, a system 410 may include the sensor system 110 which may include a humidity sensor 411. The humidity sensor 411 may detect a hydration indicator including a moisture level of the flexible elongated device 100 or the lubricious layer 104. The humidity sensor 411 may provide an electric signal based on the detected moisture level. The hydration detection system 101 may evaluate the detected moisture level to determine a hydration condition of the lubricious layer 104.

In some examples, the humidity sensor 411 may include a micro-electro-mechanical sensor (“MEMS sensor”). As shown in FIG. 6, the humidity sensor 411 may be disposed on the flexible elongated body 102 of the flexible elongated device 100. In various examples, the humidity sensor 411 may be incorporated into the lubricious layer 104, disposed under the lubricious layer 104, or deposited on an outer surface of the lubricious layer 104. The humidity sensor 411 may be disposed at a distal portion of the flexible elongated device 100, at a proximal portion of the flexible elongated device 100, or at a location between the distal and proximal portions. In some examples, a proximal end portion of the flexible elongated device 100 may be the most susceptible to dehydration. Dehydration may be more likely to occur near the proximal end portion because it is typically exposed to more air and spends more time proximal of the anatomic orifice device 206 or within portions of the anatomic orifice device 206 itself before extending into the patient anatomy. Even further, once the proximal end portion is inserted into the patient anatomy, it may be positioned in larger anatomic passageways. If the anatomic passageway is a lung airway, a large lung airway with greater airflow and less tissue contact may dehydrate the lubricious layer more quickly than portions located in smaller lung airway with less airflow.

In some examples, a plurality of the humidity sensors may be spaced radially about the flexible elongated device 100. The radial spacing may allow the humidity sensors to detect changes in hydration conditions at different radial positions around the of the flexible elongated device 100. In some examples, the hydration condition of the lubricious layer may vary by radial position. A responsive action, such as delivering a hydration fluid, may be directed to the side of the flexible elongated device that is in a sub-threshold hydrated condition and away from a side that is above the threshold hydrated condition. In some examples the humidity sensor 411 transmits electrical signals via an electrical wire or cable. However, in some examples, the humidity sensor 411 may transmit signals wirelessly. For example, the humidity sensor 411 may include a wireless transmitter for wi-fi or other wireless communication capabilities. In some examples, the humidity sensor 411 may be attached to the lubricious layer 104 or the flexible elongated body 102 using an adhesive.

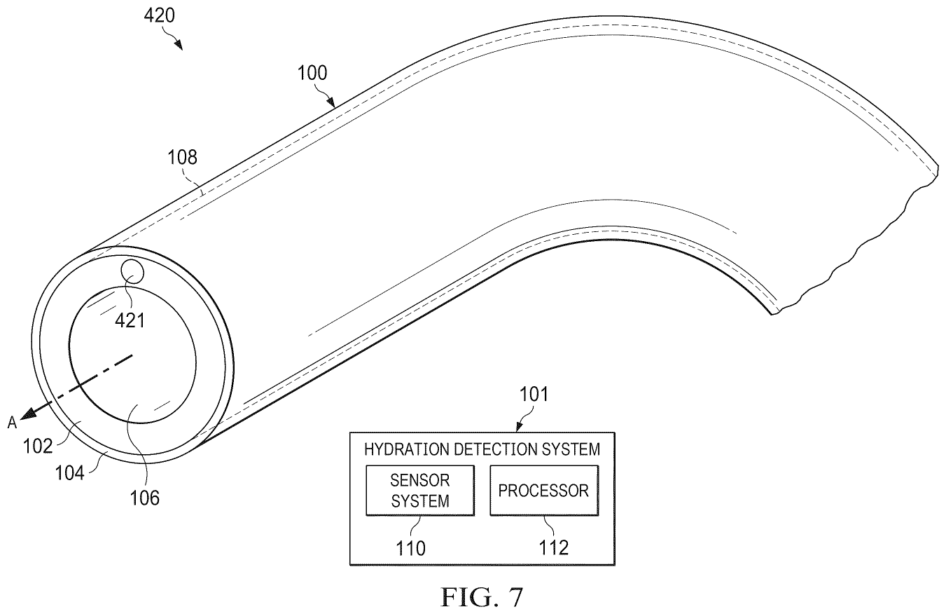

In some examples and with reference to FIG. 7, a system 420 may include the sensor system 110 which may include an imaging system 421. In some examples, the imaging system 421 may be an image capture assembly of a visualization system (e.g., a visualization system 609) of a robot-assisted medical system. In some examples, the imaging system may extend through a dedicated channel in the flexible elongated body 102, and in other examples the imaging system may extend through the lumen 106. The imaging system 421 may capture image data depicting a hydration indicator such as visible fluid, a visible fluid sheen, or other visible characteristic indicating the presence of moisture within the patient anatomy or on the flexible elongated device 100. In some examples, the imaging system 421 alone may be used to detect a hydration indicator, but in other examples may be used in combination with other sensors as described herein. In some examples, the imaging system 421 may include an endoscopic camera. As shown in FIG. 7, the imaging system 421 may include a forward-facing camera located at a distal end of the flexible elongated device 100. In other examples, the imaging system may include a side-facing camera located along a side of the flexible elongated device 100.

In some examples, the imaging system 421 may detect a visual characteristic of the anatomic passageway in which the flexible elongated device 100 is extended. For example, the imaging system 421 may generate image data which can be used to identify the cause of insertion issues as an obstruction in the anatomic passage, rather than a sub-threshold hydrated condition of the lubricious layer 104. The image data may also show visual characteristics of tissue within the anatomic passageway that may be associated with a hydration condition of the lubricious layer 104. For example, a visual sheen of the tissue in the anatomic passageway may indicate that the environment around the lubricious layer 104 is conducive to maintaining hydration of the lubricious layer. If the image data shows that the tissue is matte or dry, an anhydrous or sub-threshold hydrated condition of the lubricious layer 104 may be indicated or may soon develop.

In some examples, the imaging system 421 may be used to view the lubricious layer 104 itself, rather than visual characteristics of the anatomic passageway surrounding the lubricious layer 104. For example, the imaging system 421 may be raised relative to the lubricous layer 104, providing a view across a surface of the lubricious layer 104. The resulting image data may be analyzed to determine whether the condition of the lubricious layer 104. When the lubricous layer 104 is swollen and in a hydrated condition, the lubricious layer may appear glossy or reflective. When the lubricous layer is in a sub-threshold hydrated condition, the lubricious layer 104 may absorb light, providing a matte or dull appearance. In other examples, the imaging data may be analyzed to determine a width of the flexible elongated device 100 and/or a thickness of the lubricious layer 104 which may indicate the hydration condition.

In some examples, the sensor system 110 may include a light sensor. For example, the sensor 210 may include a light sensor configured to detect a hydration indicator. In some examples, the light sensor may include a visible light sensor. In other examples, the light sensor may include a nonvisible light sensor such as an infrared sensor. A light sensor may include a light source and a light detector. In some examples, the light source may include an optical fiber or light may be transmitted to the visible light source via an optical fiber. The light source may illuminate the flexible elongated device 100 and the lubricious layer 104. The lubricious layer 104 illuminated by the visible light source, may provide a hydration indicator in the form of a light property such as a spectral response to the light. The spectral response may be detected by the light detector. A spectral response at predetermined wavelengths may correspond to the hydration conditions, including the anhydrous hydrated condition, the threshold hydrated condition, and the fully hydrated condition. The various hydration conditions may be associated with predetermined frequency absorptions or reflections.

In some examples, a visible light sensor may include a visible laser, such as HeNe laser at approximately 633 nm, and a light detector in the form of an optical micrometer. The laser and micrometer may be used to measure and monitor a change in the diameter of the flexible elongated device 100, thus measuring and monitoring a change in the thickness of the lubricious layer 104. As previously described, a reduction in lubricious layer thickness (and consequently a reduction in diameter of the flexible elongated device) may be a hydration indicator. For example, the diameter of the flexible elongated device 100 with the lubricious layer in a fully hydrated condition may be measured by the laser and micrometer and the beginning of a procedure and a change in the diameter may be measured throughout the procedure. The measurement may be evaluated throughout the procedure to determine if the lubricious layer has reached a sub-threshold hydrated condition associated with a reduced diameter. In some examples, the thickness of the lubricious layer 104 may range from approximately 20-25 μm in a fully hydrated condition to approximately 2-3 μm in the anhydrous condition.

In some examples, an infrared light sensor may include an infrared light source and an infrared spectrometer to measure the spectral response. The infrared spectrometer may be used to detect various wavelengths (or frequencies) of infrared light reflected from the lubricious layer 104 at different hydration conditions. Different hydration conditions may be associated with different frequencies of absorption or reflection based on the concentration of moisture (oxygen-hydrogen bonds) within the lubricious layer. For example, the presence of water may cause the lubricious layer 104 to absorb more or certain frequencies of infrared light. In contrast, when the lubricious layer is dehydrated, it may reflect more infrared light and absorb less or specific wavelengths of light. The infrared light range of interest may be between approximately 2700 nm and 3200 nm which correspond to the hydroxyl group (—OH) in water. In a fully hydrated condition, the light absorbance by the lubricious layer 104 may be 2-3 times as great as the light absorbance in the anhydrous condition. Thus a measured light absorption that is substantially lower than the light absorption at an initial, fully hydrated condition may be an indicator that the lubricious layer has reached a sub-threshold hydrated condition.

In some examples, a light sensor may be located on the inside of the anatomic orifice device 206 or may view the flexible elongated device 100 through a window of the anatomic orifice device. In other examples, the light sensor may be located at a more remote location, and the delivered and reflected light may be transmitted by an optical fiber.

In some examples, the sensor system 110 may include a vibration sensor. For example, the sensor 210 may include a vibration or auditory sensor configured to detect a hydration indicator. The vibration sensor may detect vibration noise created by contact, such as frictional contact, with a component of the medical system through which the flexible elongated device 100 is passing. For example, when the flexible elongated device 100 is inserted through the anatomic orifice device 206, the lubricious layer 104 may rub the inner walls of the anatomic orifice device 206. The vibration sensor may detect the noise as a hydration indicator of the lubricious layer 104. In some examples, the lubricious layer 104 in an anhydrous condition rubbing the anatomic orifice device 206 or the anatomic passageways may generate more vibration noise than the lubricious layer 104 in a fully hydrated condition. The vibration noise created by the friction may be a hydration indicator for the lubricious layer 104.

In some examples, the vibration sensor 412 may be a microphone or any other noise sensing device. The vibration sensor may be located on the anatomic orifice device 206 but may, alternatively, be located elsewhere in the environment of the flexible elongated device 100, including on the flexible elongated device 100, on the manipulator assembly 200, or on the carriage 202.

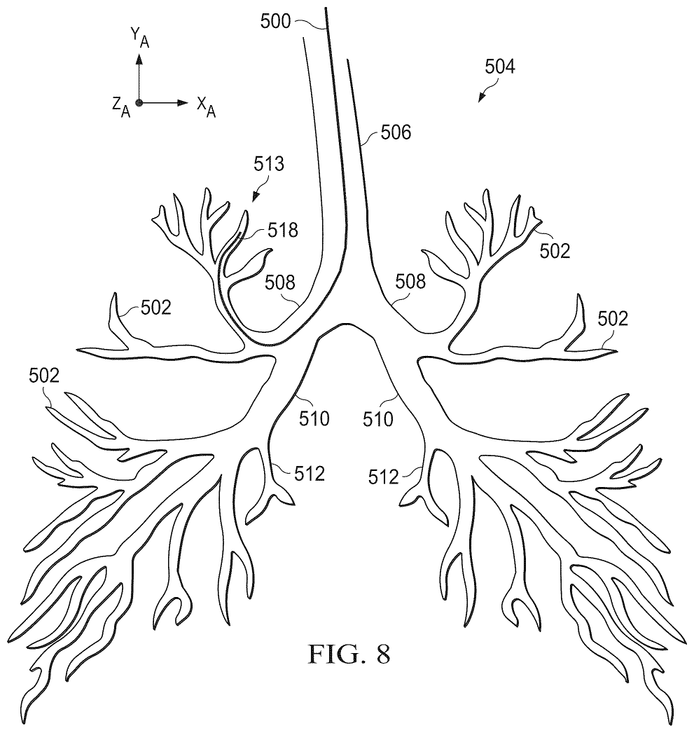

FIG. 8 illustrates a flexible elongated device 500 (e.g. flexible elongated device 100) extending within branched anatomic passageways or airways 502 of an anatomical structure 504. In some examples the anatomic structure 504 may be a lung and the passageways 502 may include the trachea 506, primary bronchi 508, secondary bronchi 510, and tertiary bronchi 512. The anatomic structure 504 has an anatomical frame of reference (X_{A}, Y_{A}, Z_{A}). A distal end portion 518 of the flexible elongated device 500 may be advanced into an anatomic opening (e.g., a patient mouth) and through the anatomic passageways 502 to perform a medical procedure, such as a biopsy, ablation, electroporation, or other type of diagnostic or therapeutic procedure, at or near a target tissue 513. The flexible elongated device 500 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose robot-assisted medical systems.

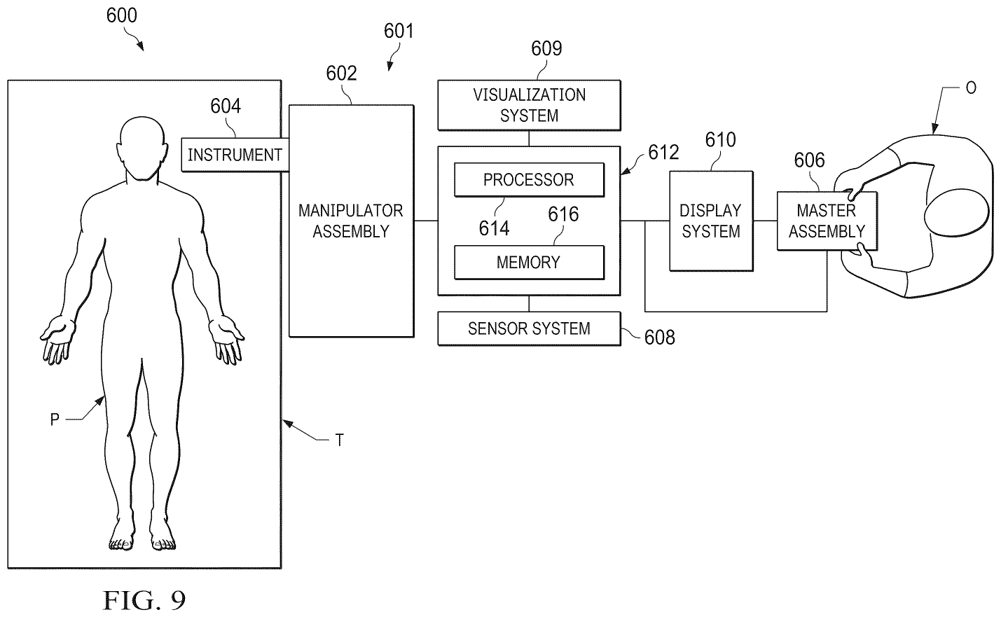

FIG. 9 illustrates a medical system 600 that may include a manipulator assembly 602 that controls the operation of a medical instrument 604 such as a flexible elongated device (e.g., a flexible elongated device 100) in performing various procedures on a patient P. Medical instrument 604 may extend into an internal site within the body of patient P via an opening in the body of patient P. The manipulator assembly 602 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. The manipulator assembly 602 may be mounted to and/or positioned near a patient table T. A master assembly 606 allows an operator O (e.g., a surgeon, a clinician, a physician, or other user) to control the manipulator assembly 602. In some examples, the master assembly 606 allows the operator O to view the procedural site or other graphical or informational displays. In some examples, the manipulator assembly 602 may be excluded from the medical system 600 and the instrument 604 may be controlled directly by the operator O. In some examples, the manipulator assembly 602 may be manually controlled by the operator O. Direct operator control may include various handles and operator interfaces for hand-held operation of the instrument 604.

The master assembly 606 may be located at a surgeon's console which is in proximity to (e.g., in the same room as) a patient table T on which patient P is located, such as at the side of the patient table T. In some examples, the master assembly 606 is remote from the patient table T, such as in in a different room or a different building from the patient table T. The master assembly 606 may include one or more control devices for controlling the manipulator assembly 602. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 602 supports the medical instrument 604 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 612). The manipulator assembly 602 may include a plurality of actuators (e.g., motors) that drive inputs on the medical instrument 604 in response to commands, such as from the control system 612. The actuators may include drive systems that move the medical instrument 604 in various ways when coupled to the medical instrument 604. For example, one or more actuators may advance medical instrument 604 into a naturally or surgically created anatomic orifice. Actuators may control articulation of the medical instrument 604, such as by moving the distal end (or any other portion) of medical instrument 604 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 604, such as for grasping tissue in the jaws of a biopsy device and/or the like or may be used to move or otherwise control tools (e.g., imaging tools, ablation tools, biopsy tools, electroporation tools, etc.) that are inserted within the medical instrument 604.

The medical system 600 may include a sensor system 608 with one or more sub-systems for receiving information about the manipulator assembly 602 and/or the medical instrument 604. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 604; a visualization system 609 (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 604 or from some other location; and/or actuator position sensors such as resolvers, encoders, potentiometers, and the like that describe the rotation and/or orientation of the actuators controlling the medical instrument 604.

The medical system 600 may include a display system 610 for displaying an image or representation of the procedural site and the medical instrument 604. Display system 610 and master assembly 606 may be oriented so physician O can control medical instrument 604 and master assembly 606 with the perception of telepresence.

In some embodiments, the medical instrument 604 may include a visualization system 609, which may include an image capture assembly that records a concurrent or real-time image of a procedural site and provides the image to the operator O through one or more displays of display system 610. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 604. Additionally or alternatively, a separate endoscope, attached to a separate manipulator assembly, may be used with medical instrument 604 to image the procedural site. The visualization system may be implemented as hardware, firmware, software or a combination thereof which interact with or are otherwise executed by one or more computer processors, such as of the control system 612.

Display system 610 may also display an image of the procedural site and medical instruments, which may be captured by the visualization system. In some examples, the medical system 600 provides a perception of telepresence to the operator O. For example, images captured by an imaging device at a distal portion of the medical instrument 604 may be presented by the display system 610 to provide the perception of being at the distal portion of the medical instrument 604 to the operator O. The input to the master assembly 606 provided by the operator O may move the distal portion of the medical instrument 604 in a manner that corresponds with the nature of the input (e.g., distal tip turns right when a trackball is rolled to the right) and results in corresponding change to the perspective of the images captured by the imaging device at the distal portion of the medical instrument 604. As such, the perception of telepresence for the operator O is maintained as the medical instrument 604 is moved using the master assembly 606. The operator O can manipulate the medical instrument 604 and hand controls of the master assembly 606 as if viewing the workspace in substantially true presence, simulating the experience of an operator that is physically manipulating the medical instrument 604 from within the patient anatomy.

In some examples, the display system 610 may present virtual images of a procedural site that are created using image data recorded pre-operatively or intra-operatively, such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 610 may display a virtual image that is generated based on tracking the location of medical instrument 604. For example, the tracked location of the medical instrument 604 may be registered (e.g., dynamically referenced) with the model generated using the pre-operative or intra-operative images, with different portions of the model correspond with different locations of the patient anatomy. As the medical instrument 604 moves through the patient anatomy, the registration is used to determine portions of the model corresponding with the location and/or perspective of the medical instrument 604 and virtual images are generated using the determined portions of the model. This may be done to present the operator O with virtual images of the internal procedural site from viewpoints of medical instrument 604 that correspond with the tracked locations of the medical instrument 604.

The medical system 600 may also include the control system 612, which may include processing circuitry that implements the some or all of the methods or functionality discussed herein. The control system 612 may include at least one memory 816 and at least one processor 614 for controlling the operations of the manipulator assembly 602, the medical instrument 604, the master assembly 606, the sensor system 608, and/or the display system 610. Control system 612 may include instructions (e.g., a non-transitory machine-readable medium storing the instructions) that when executed by the at least one processor, configures the one or more processors to implement some or all of the methods or functionality discussed herein. While the control system 612 is shown as a single block in FIG. 9, the control system 612 may include two or more separate data processing circuits with one portion of the processing being performed at the manipulator assembly 602, another portion of the processing being performed at the master assembly 606, and/or the like. In some examples, the control system 612 may include other types of processing circuitry, such as application-specific integrated circuits (ASICs) and/or field-programmable gate array (FPGAs). The control system 612 may be implemented using hardware, firmware, software, or a combination thereof.

In some examples, the control system 612 may receive feedback from the medical instrument 604, such as force and/or torque feedback. Responsive to the feedback, the control system 612 may transmit signals to the master assembly 606. In some examples, the control system 612 may transmit signals instructing one or more actuators of the manipulator assembly 602 to move the medical instrument 604. In some examples, the control system 612 may transmit informational displays regarding the feedback to the display system 610 for presentation or perform other types of actions based on the feedback.

The control system 612 may include a virtual visualization system to provide navigation assistance to operator O when controlling the medical instrument 604 during an image-guided medical procedure. Virtual navigation using the virtual visualization system may be based upon an acquired pre-operative or intra-operative dataset of anatomic passageways of the patient P. The control system 612 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 608 that is used to compute an (e.g., approximate) location of the medical instrument 604 with respect to the anatomy of patient P. The sensor system 608 may be used to register and display the medical instrument 604 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 608 may be used to compute the (e.g., approximate) location of the medical instrument 604 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 600 may further include operations and support systems (not shown) such as illumination systems, steering control systems, irrigation systems, and/or suction systems. In some embodiments, the medical system 600 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

FIG. 10A is a simplified diagram of a medical instrument system 700 according to some embodiments. The medical instrument system 700 includes a flexible elongated device 702 (e.g. flexible elongated device), a drive unit 704, and a medical tool 726 that collectively is an example of a medical instrument 604 of a medical system 600. The medical system 600 may be a robot-assisted system, a non-robot-assisted system, or a hybrid robot-assisted and non-assisted system, as explained with reference to FIG. 9. A visualization system 731, tracking system 730, and navigation system 732 are also shown in FIG. 10A and are example components of the control system 612 of the medical system 600. In some examples, the medical instrument system 700 may be used for non-robot-assisted exploratory procedures or in procedures involving traditional manually operated medical instruments, such as endoscopy. The medical instrument system 700 may be used to gather (e.g., measure) a set of data points corresponding to locations within anatomic passageways of a patient, such as patient P.

The elongated device 702 is coupled to the drive unit 704. The elongated device 702 includes a lumen or channel 721 through which the medical tool 726 may be inserted. The elongated device 702 navigates within patient anatomy to deliver the medical tool 726 to a procedural site. The elongated device 702 includes a flexible body 716 having a proximal end 717 and a distal end 718. In some examples, the flexible body 716 may have an approximately 3 mm outer diameter. Other flexible body outer diameters may be larger or smaller.

Medical instrument system 700 may include the tracking system 730 for determining the position, orientation, speed, velocity, pose, and/or shape of the flexible body 716 at the distal end 718 and/or of one or more segments 724 along flexible body 716, as will be described in further detail below. The tracking system 730 may include one or more sensors and/or imaging devices. The flexible body 716, such as the length between the distal end 718 and the proximal end 717, may include multiple segments 724. The tracking system 730 may be implemented using hardware, firmware, software, or a combination thereof. In some examples, the tracking system 730 is part of control system 712.

Tracking system 730 may track the distal end 718 and/or one or more of the segments 724 of the flexible body 716 using a shape sensor 722. The shape sensor 722 may include an optical fiber aligned with the flexible body 716 (e.g., provided within an interior channel of the flexible body 716 or mounted externally along the flexible body 716). In some examples, the optical fiber may have a diameter of approximately 200 μm. In other examples, the diameter may be larger or smaller. The optical fiber of the shape sensor 722 may form a fiber optic bend sensor for determining the shape of flexible body 716. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 716 may be determined using other techniques. For example, a history of the position and/or pose of the distal end 718 of the flexible body 716 can be used to reconstruct the shape of flexible body 716 over an interval of time (e.g., as the flexible body 716 is advanced or retracted within a patient anatomy). In some examples, the tracking system 730 may alternatively and/or additionally track the distal end 718 of the flexible body 716 using a position sensor system 720. Position sensor system 720 may be a component of an EM sensor system with the position sensor system 720 including one or more position sensors. Although the position sensor system 720 is shown as being near the distal end 718 of the flexible body 716 to track the distal end 718, the number and location of the position sensors of the position sensor system 720 may vary to track different regions along the flexible body 716. In one example, the position sensors include conductive coils that may be subjected to an externally generated electromagnetic field. Each coil of position sensor system 720 may produce an induced electrical signal having characteristics that depend on the position and orientation of the coil relative to the externally generated electromagnetic field. The position sensor system 720 may measure one or more position coordinates and/or one or more orientation angles associated with one or more portions of flexible body 716. In some examples, the position sensor system 720 may be configured and positioned to measure six degrees of freedom, e.g., three position coordinates X, Y, Z and three orientation angles indicating pitch, yaw, and roll of a base point. In some examples, the position sensor system 720 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 730 may alternately and/or additionally rely on a collection of pose, position, and/or orientation data stored for a point of an elongated device 702 and/or medical tool 726 captured during one or more cycles of alternating motion, such as breathing. This stored data may be used to develop shape information about the flexible body 916. In some examples, a series of position sensors (not shown), such as EM sensors like the sensors in position sensor 720 or some other type of position sensors may be positioned along the flexible body 716 and used for shape sensing. In some examples, a history of data from one or more of these position sensors taken during a procedure may be used to represent the shape of elongated device 702, particularly if an anatomic passageway is generally static.

FIG. 10B is a simplified diagram of the medical tool 726 within the elongated device 702 according to some embodiments. The flexible body 716 of the elongated device 702 may include the channel 721 sized and shaped to receive the medical tool 726. In some embodiments, the medical tool 726 may be used for procedures such as diagnostics, imaging, surgery, biopsy, ablation, illumination, irrigation, suction, electroporation, etc. Medical tool 726 can be deployed through channel 721 of flexible body 716 and operated at a procedural site within the anatomy. Medical tool 726 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 926 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 726 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 721 when the biopsy tool is within the channel 721. The medical tool 726 may be an image capture probe that includes a distal portion with a stereoscopic or monoscopic camera that may be placed at or near the distal end 718 of flexible body 916 for capturing images (e.g., still or video images). The captured images may be processed by the visualization system 731 for display and/or provided to the tracking system 730 to support tracking of the distal end 718 of the flexible body 716 and/or one or more of the segments 724 of the flexible body 716. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 731. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 716 of the elongated device 702 to facilitate visual navigation of the elongated device 702 to a procedural site and then is replaced within the flexible body 716 with another type of medical tool 726 that performs the procedure. In some examples, the image capture probe may be within the flexible body 716 of the elongated device 702 along with another type of medical tool 726 to facilitate simultaneous image capture and tissue intervention, such as within the same channel 721 or in separate channels. A medical tool 726 may be advanced from the opening of the channel 721 to perform the procedure (or some other functionality) and then retracted back into the channel 721 when the procedure is complete. The medical tool 726 may be removed from the proximal end 717 of the flexible body 716 or from another optional instrument port (not shown) along flexible body 716.

In some examples, the elongated device 702 may include integrated imaging capability rather than utilize a removable image capture probe. For example, the imaging device (or fiber-optic bundle) and the light emitters may be located at the distal end 718 of the elongated device 702. The flexible body 715 may include one or more dedicated channels that carry the cable(s) and/or optical fiber(s) between the distal end 718 and the visualization system 731. Here, the medical instrument system 700 can perform simultaneous imaging and tool operations.

In some examples, the medical tool 726 is capable of controllable articulation. The medical tool 726 may house control members or cables (which may also be referred to as pull wires), linkages, or other actuation controls (not shown) that extend between its proximal and distal ends to controllably bend the distal end of medical tool 726, such as discussed herein for the flexible elongated device 702. The medical tool 726 may be coupled to a drive unit 704 and the manipulator assembly 602. In these examples, the elongated device 702 may be excluded from the medical instrument system 700 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sep. 30, 2008 and titled “Passive Preload and Capstan Drive for Surgical Instruments”), which are incorporated by reference herein in their entireties.

The flexible body 716 of the elongated device 702 may also or alternatively house cables, linkages, or other steering controls (not shown) that extend between the drive unit 704 and the distal end 718 to controllably bend the distal end 718 as shown, for example, by broken dashed line depictions 719 of the distal end 918 in FIG. 10A. In some examples, at least four cables are used to provide independent up-down steering to control a pitch of the distal end 718 and left-right steering to control a yaw of the distal end 718. In these examples, the flexible elongated device 702 may be a steerable catheter. Examples of steerable catheters, applicable in some embodiments, are described in detail in PCT Publication WO 2019/018736 (published Jan. 24, 2019 and titled “Flexible Elongated Device Systems and Methods”), which is incorporated by reference herein in its entirety.

In embodiments where the device 702 and/or medical tool 726 are actuated by a robot-assisted assembly (e.g., the manipulator assembly 602), the drive unit 704 may include drive inputs that removably couple to and receive power from drive elements, such as actuators, of the robot-assisted assembly. In some examples, the elongated device 702 and/or medical tool 726 may include gripping features, manual actuators, or other components for manually controlling the motion of the elongated device 702 and/or medical tool 726. The elongated device 702 may be steerable or, alternatively, the elongated device 702 may be non-steerable with no integrated mechanism for operator control of the bending of distal end 718. In some examples, one or more channels 721 (which may also be referred to as lumens), through which medical tools 726 can be deployed and used at a target anatomical location, may be defined by the interior walls of the flexible body 716 of the elongated device 702.

In some examples, the medical instrument system 700 (e.g., the elongated device 702 or medical tool 726) may include a flexible bronchial instrument, such as a bronchoscope or bronchial catheter, for use in examination, diagnosis, biopsy, and/or treatment of a lung. The medical instrument system 700 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 730 may be sent to the navigation system 732, where the information may be combined with information from the visualization system 731 and/or pre-operatively obtained models to provide the physician, clinician, surgeon, or other operator with real-time position information. In some examples, the real-time position information may be displayed on the display system 610 for use in the control of the medical instrument system 700. In some examples, the navigation system 732 may utilize the position information as feedback for positioning medical instrument system 700. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

The methods described herein are illustrated as a set of operations or processes. Not all the illustrated processes may be performed in all examples of the methods. Additionally, one or more processes that are not expressly illustrated or described may be included before, after, in between, or as part of the example processes. In some examples, one or more of the processes may be performed by the control system (e.g., control system 612) or may be implemented, at least in part, in the form of executable code stored on non-transitory, tangible, machine-readable media that when run by one or more processors (e.g., the processors 614 of control system 612) may cause the one or more processors to perform one or more of the processes.

One or more components of the embodiments discussed in this disclosure, such as control system 612, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

In some instances well known methods, procedures, components, and circuits have not been described in detail so as not to unnecessarily obscure aspects of the examples. This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

While certain exemplary examples of the invention have been described and shown in the accompanying drawings, it is to be understood that such examples are merely illustrative of and not restrictive on the broad invention, and that the examples of the invention not be limited to the specific constructions and arrangements shown and described, since various other alternatives, modifications, and equivalents will be appreciated by those with ordinary skill in the art.

1. A system comprising:
a flexible elongated device including a flexible elongated body and a hydrophilic lubricious layer on the flexible elongated body;
a sensor system configured to detect a hydration indicator for the hydrophilic lubricious layer; and
a control system configured to evaluate the hydration indicator to determine a hydration condition of the hydrophilic lubricious layer.

2. The system of claim 1, wherein the control system is further configured to initiate a responsive action if the hydration condition is determined to be below a threshold hydrated condition.

3. The system of claim 2, wherein the responsive action is an issuance of an alert.

4. The system of claim 2, wherein the responsive action is a delivery of hydration to the flexible elongated device.

5. The system of claim 2, wherein the responsive action is an initiation of a change of motion of the flexible elongated device.

6. The system of claim 1, wherein the sensor system includes a timer configured to detect an elapsed time that the flexible elongated device has been in an exposed environment and wherein the hydration indicator includes the elapsed time.

7. The system of claim 6, wherein evaluating the hydration indicator includes evaluating the elapsed time with reference to a time-based dehydration model.

8. The system of claim 1, wherein the sensor system includes a force sensor configured to detect friction force data for the flexible elongated device and wherein hydration indicator includes the friction force data.

9. The system of claim 8, wherein evaluating the hydration indicator includes recognizing a friction force pattern in the friction force data, wherein the friction force pattern is associated with the hydration condition.

10. The system of claim 1, wherein the sensor system includes a shape sensor disposed in the flexible elongated device and configured to detect a shape of the flexible elongated device, and wherein the hydration indicator includes the detected shape.

11. The system of claim 10, wherein evaluating the hydration indicator includes detecting a prolapse shape in the detected shape, wherein the prolapse shape is associated with the hydration condition.

12. The system of claim 1, wherein the sensor system includes a sensor configured to detect a hydrochromic pigment on the flexible elongated device and wherein the hydration indicator includes a color property of the hydrochromic pigment associated with hydration.

13. The system of claim 12, wherein evaluating the hydration indicator includes determining the color property of the hydrochromic pigment is associated with the hydration condition.

14. The system of claim 12, wherein the hydrochromic pigment is incorporated into the hydrophilic lubricious layer.

15. The system of claim 1, wherein the sensor system includes a humidity sensor configured to detect a moisture level of the flexible elongated device and wherein the hydration indicator includes the moisture level.

16-18. (canceled)

19. The system of claim 1, wherein the sensor system includes an imaging system configured to capture an image of the hydration indicator.

20. The system of claim 19, wherein the hydration indicator is a visual characteristic of an anatomic passageway in which the flexible elongated device is extended.

21. The system of claim 19, wherein the hydration indicator is a visual characteristic of the hydrophilic lubricious layer.

22. The system of claim 1, wherein the sensor system includes a light sensor configured to detect a light property of the flexible elongated device associated with the hydration indicator.

23-24. (canceled)

25. The system of claim 1, wherein the sensor system includes a vibration sensor configured to detect a sound associated with the flexible elongated device, and wherein the hydration indicator includes the detected sound.

26. (canceled)

27. A method comprising:
detecting, with a sensor system, a hydration indicator for a hydrophilic lubricious layer on a flexible elongated body of a flexible elongated device; and
evaluating, with a control system, the hydration indicator to determine a hydration condition for the hydrophilic lubricious layer.

28. The method of claim 27, further comprising initiating a responsive action if the hydration condition is determined to be below a threshold hydrated condition.

29-36. (canceled)
